(19) United States
(12) Patent Application Publication
(10) Pub. No.: US 2026/0315177 A1
(43) Pub. Date: Oct. 15, 2026

(54) Adjusting Illumination Sources Based on Stages of Ophthalmic Surgery

(71) Applicant: Alcon Inc., Fribourg (CH)

(72) Inventors: John Park, Irvine, CA (US); Ronald T. Smith, Irvine, CA (US); Lingfeng Yu, Rancho Santa Margarita, CA (US)

(21) Appl. No.: 19/466,651

(22) Filed: Feb. 2, 2026

Related U.S. Application Data
(60) Provisional application No. 63/753,770, filed on Feb. 4, 2025.

(51) Int. Cl. A61B 90/30; A61B 90/00; A61F 2/16; A61F 9/007; G16H 20/40; G16H 40/63

(52) U.S. Cl. CPC A61B 90/30; A61B 90/361; A61F 2/1662; A61F 9/00745; A61F 9/00781; G16H 20/40; G16H 40/63

(57) ABSTRACT

Adjusting illumination sources based on stages of ophthalmic surgery is described. An ophthalmic surgical system may include a first illumination source configured to produce a first light beam for illuminating a surgical region of an eye, upon which an ophthalmic surgical procedure is being performed, from a first direction, and a second illumination source configured to produce a second light beam for illuminating the surgical region of the eye from a second direction. The ophthalmic surgical system may further include a surgical stage-based illumination module implemented in a non-transitory computer-readable storage medium and configured to determine, in real-time, a stage of the ophthalmic surgical procedure based on a detected trigger event. The surgical stage-based illumination module may be further configured to output, in real-time, instructions to adjust at least one of the first illumination source or the second illumination source based on the stage of the ophthalmic surgical procedure.

### BACKGROUND

A variety of diseases or conditions associated with an eye may be treated through ophthalmic surgical procedures. Examples of ophthalmic surgical procedures include cataract surgery, glaucoma surgery, laser eye surgery (LASIK), and the like. For example, cataract surgery involves emulsifying a crystalline lens with an ultrasonic handpiece and aspirating it from the eye. An intraocular lens (IOL) is then implanted in the posterior lens capsule of the eye. During various ophthalmic surgical procedures such as those mentioned above, improper surgical lighting may negatively impact the outcome, efficiency, and effectiveness of the surgery.

### SUMMARY

Adjusting illumination sources based on stages of ophthalmic surgery is described. An ophthalmic surgical system may include a camera to obtain image feed of a surgical region of an eye, upon which an ophthalmic surgical procedure is being performed, a first illumination source configured to produce a first light beam for illuminating the surgical region of the eye from a first direction, and a second illumination source configured to produce a second light beam for illuminating the surgical region of the eye from a second direction. The ophthalmic surgical system may further include a surgical stage-based illumination module implemented in a non-transitory computer-readable storage medium and configured to determine, in real-time, a stage of the ophthalmic surgical procedure based on a detected trigger event. The surgical stage-based illumination module may be further configured to output, in real-time, instructions to adjust at least one of the first illumination source or the second illumination source based on the stage of the ophthalmic surgical procedure.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures. Entities represented in the figures are indicative of one or more entities and thus, reference is made interchangeably to single or plural forms of the entities in the discussion.

FIG. 1 is an illustration of an environment in an example implementation that is operable for adjusting illumination sources based on stages of ophthalmic surgery as described herein.

FIG. 2 depicts a cross-section of an eye.

FIG. 3 depicts an example implementation of the surgical stage-based illumination module of FIG. 1 in greater detail.

FIG. 4 depicts a non-limiting example of illumination sources used in the context of an ophthalmic surgical procedure.

FIG. 5 depicts a non-limiting illustrative example overview of adjusting illumination sources based on stages of an ophthalmic surgical procedure.

FIG. 6 depicts a non-limiting illustrative example of adjusting illumination sources based on a capsulorhexis stage of cataract surgery.

FIG. 7 depicts a non-limiting illustrative example of adjusting illumination sources based on a phacoemulsification stage of cataract surgery.

FIG. 8 depicts a non-limiting illustrative example of adjusting illumination sources based on a post-phacoemulsification stage of cataract surgery.

FIG. 9 depicts a non-limiting illustrative example of adjusting illumination sources based on a lens implantation stage of cataract surgery.

FIG. 10 depicts a non-limiting illustrative example of adjusting illumination sources based on a stage of glaucoma surgery.

FIG. 11 depicts an example procedure in which adjusting illumination sources based on stages of ophthalmic surgery is performed.

FIG. 12 depicts an example procedure in which adjusting illumination sources based on stages of ophthalmic surgery is performed during a cataract surgery.

FIG. 13 illustrates an example system including various components of an example device that can be implemented as any type of computing device as described and/or utilized with reference to FIGS. 1-12 to implement the techniques described herein.

### DETAILED DESCRIPTION

### Overview

Eye surgeons operate surgical instruments inside a patient’s eye with the aid of visualization systems that provide digitally enhanced views of the eye and positions of the surgical instruments therein. The visualization systems, such as microscopes, are generally configured with multiple illumination sources to illuminate a surgical region of the patient’s eye. Different types of illumination are optimal for different stages of ophthalmic surgery, resulting in the eye surgeons manually toggling between the different types of illumination during the surgery. However, this wastes operating time, increases distractions, and introduces human-caused error into the surgical procedure.

Accordingly, adjusting illumination sources based on stages of ophthalmic surgery is described herein. By way of example, an ophthalmic surgical system automatically detects a surgical stage based on a trigger event and identifies an illumination adjustment in real-time that corresponds to the surgical stage. This addresses the challenges of conventional ophthalmic surgical illumination systems because the eye surgeon is free to perform multiple stages of the ophthalmic surgical procedure in sequence without interruptions, while the illumination sources automatically update to optimal parameters according to current stages of the ophthalmic surgical procedure. This can save the surgeon time, can reduce distractions, and/or can reduce instances of human-caused error related to illumination sources during the ophthalmic surgical procedure.

In accordance with the described techniques, the ophthalmic surgical system includes an intra-operative imaging system, for example, a microscope equipped with a camera for capturing live image feed of a surgical region of the patient’s eye. The ophthalmic surgical system also includes at least a first illumination source configured to project an oblique beam of light directed at an angle toward the cornea of the eye, and a second illumination source configured to project multiple coaxial beams of light directed straight toward the cornea of the eye. The first illumination source and the second illumination source, for instance, are configurable to adjust various illumination properties related to the oblique beam and the coaxial beams, including, but not limited to, light color temperature and light illuminance.

The ophthalmic surgical system begins in this example by identifying a stage of the ophthalmic surgical procedure based on a detected trigger event. In an example implementation, the ophthalmic surgical system may leverage an instrument identification algorithm trained to identify an instrument in a frame of the image feed that indicates occurrence of a specific stage of an ophthalmic surgical procedure, using computer vision techniques. For instance, the instrument identification system may identify usage of a phacoemulsification instrument, indicating occurrence of a phacoemulsification stage of a cataract surgery procedure. Additionally or alternatively, the ophthalmic surgical system may leverage an algorithm trained to identify other actions or objects in frames of the image feed that indicate occurrence of the specific stage of the ophthalmic surgical procedure.

In another example implementation, the ophthalmic surgical system receives data indicating actuation of the instrument and therefore identifies occurrence of the specific stage of the ophthalmic surgical procedure. For example, the ophthalmic surgical system receives data indicating that the phacoemulsification instrument has been actuated and therefore determines that the phacoemulsification stage of the cataract surgery is underway.

Based on the identified stage of the ophthalmic surgical procedure, the ophthalmic surgical system outputs instructions in real-time to adjust the first illumination source and/or the second illumination source. This is because specific illumination adjustments are optimal for specific stages of ophthalmic surgical procedures. For example, an increase in red light for the coaxial beams is optimal during the phacoemulsification stage of the cataract surgery, in which the surgeon breaks up and removes cataract material. This is because the red light increases brightness of the red reflex, which is a red glow that appears when light reflects off the retina at the back of the eye. The red reflex provides visual contrast that helps the surgeon identify the boundaries of the cataract and surrounding structures, guiding precise instrument placement and movement. The instructions in this example therefore indicate an adjustment to light color temperature by increasing warm light and decreasing cool light of the illumination sources to increase the brightness of the red reflex.

Other examples of illumination adjustments include a decrease to the warm light and a corresponding increase to the cool light, an increase to light intensity, a decrease to light intensity, and actuation of the oblique beam and/or the coaxial beams on or off using the illumination sources. For instance, the warm light and the cool light are adjusted by tuning red, green, and blue light values of the illumination sources.

Furthermore, the techniques described herein are adaptable to various ophthalmic surgical procedures, including cataract surgery, glaucoma surgery, LASIK surgery, retina surgery, or other ophthalmic surgical procedures. For instance, the described techniques for adjusting illumination sources based on stages of ophthalmic surgery can be configured to detect surgical stage based on a trigger event and identify an illumination adjustment in real-time that corresponds to the surgical stage. This flexibility, combined with the real-time capabilities of the described approach, enables precise illumination across a range of ophthalmic applications.

In some aspects, the techniques described herein relate to an ophthalmic surgical system including: a camera to obtain image feed of a surgical region of an eye, upon which an ophthalmic surgical procedure is being performed, a first illumination source configured to produce a first light beam for illuminating the surgical region of the eye from a first direction, a second illumination source configured to produce a second light beam for illuminating the surgical region of the eye from a second direction, and a surgical stage-based illumination module implemented in a non-transitory computer-readable storage medium and configured to perform operations including: determining, in real-time, a stage of the ophthalmic surgical procedure based on a detected trigger event, and outputting, in real-time, instructions to adjust at least one of the first illumination source or the second illumination source based on the stage of the ophthalmic surgical procedure.

In some aspects, the techniques described herein relate to an ophthalmic surgical system, wherein the first light beam is an oblique beam directed at a diagonal angle toward a cornea of an eye.

In some aspects, the techniques described herein relate to an ophthalmic surgical system, wherein the second light beam includes two coaxial beams directed at a perpendicular angle toward a cornea of an eye, relative to a corneal plane of the eye.

In some aspects, the techniques described herein relate to an ophthalmic surgical system, wherein the detected trigger event includes actuation of an instrument associated with performing the stage of the ophthalmic surgical procedure.

In some aspects, the techniques described herein relate to an ophthalmic surgical system, wherein: the ophthalmic surgical procedure is cataract surgery, the detected trigger event includes creation of an incision into a cornea of an eye, and the surgical stage-based illumination module is further configured to tune a color of at least the first illumination source or the second illumination source to increase blue light during a capsulorhexis stage of the ophthalmic surgical procedure.

In some aspects, the techniques described herein relate to an ophthalmic surgical system, wherein: the ophthalmic surgical procedure is cataract surgery, the detected trigger event includes actuation of a phacoemulsification instrument for phacoemulsification, and the surgical stage-based illumination module is further configured to tune a color of at least the first illumination source or the second illumination source to increase red content of light during a phacoemulsification stage of the ophthalmic surgical procedure.

In some aspects, the techniques described herein relate to an ophthalmic surgical system, wherein: the ophthalmic surgical procedure is cataract surgery, the detected trigger event includes removal of a lens of an eye during phacoemulsification, and the surgical stage-based illumination module is further configured to tune a color of at least the first illumination source or the second illumination source to reduce blue light following a phacoemulsification stage of the ophthalmic surgical procedure.

In some aspects, the techniques described herein relate to an ophthalmic surgical system, wherein: the ophthalmic surgical procedure is cataract surgery, the detected trigger event includes actuation of a lens implantation instrument, and the surgical stage-based illumination module is further configured to tune a color of at least the first illumination source or the second illumination source to a preconfigured intensity or color during a lens implantation stage of the ophthalmic surgical procedure.

In some aspects, the techniques described herein relate to an ophthalmic surgical system, wherein the surgical stage-based illumination module is configured to determine the detected trigger event based on the image feed of the ophthalmic surgical procedure.

In some aspects, the techniques described herein relate to an ophthalmic surgical system, wherein: the ophthalmic surgical procedure is glaucoma surgery, the detected trigger event includes detected use of a stent implanter instrument, and the surgical stage-based illumination module is further configured to actuate the first illumination source, which is an oblique beam, during a stent implantation stage of the ophthalmic surgical procedure.

In some aspects, the techniques described herein relate to an ophthalmic surgical system, wherein the surgical stage-based illumination module is configured to adjust a warmth of a color of the first illumination source or the second illumination source by tuning colors of the first illumination source or the second illumination source based on the stage of the ophthalmic surgical procedure.

In some aspects, the techniques described herein relate to an ophthalmic surgical system, wherein the surgical stage-based illumination module is configured to tune an intensity of the first illumination source or the second illumination source based on the stage of the ophthalmic surgical procedure.

In some aspects, the techniques described herein relate to a method for an ophthalmic surgical procedure, including: receiving, via a surgical stage-based detection system, information indicating occurrence of a trigger event during the ophthalmic surgical procedure, determining, in real-time, a stage of the ophthalmic surgical procedure based on the trigger event, and outputting, in real-time, instructions to adjust an oblique beam of an illumination device or coaxial beams of the illumination device based on the stage of the ophthalmic surgical procedure.

In some aspects, the techniques described herein relate to a method, wherein the trigger event includes actuation of an instrument for performing the stage of the ophthalmic surgical procedure.

In some aspects, the techniques described herein relate to a method, further including determining the trigger event based on received image feed of the ophthalmic surgical procedure.

In some aspects, the techniques described herein relate to a method, further including adjusting a warmth of a color of the oblique beam of the illumination device or the coaxial beams of the illumination device by tuning colors of the oblique beam of the illumination device or the coaxial beams of the illumination device based on the stage of the ophthalmic surgical procedure.

In some aspects, the techniques described herein relate to a method, further including adjusting a level of zoom of image feed of the ophthalmic surgical procedure based on the stage of the ophthalmic surgical procedure.

In some aspects, the techniques described herein relate to an ophthalmic surgical system including: a first illumination source configured to produce an oblique beam for illuminating a surgical region of an eye, upon which an ophthalmic surgical procedure is being performed, a second illumination source configured to produce coaxial beams for illuminating the surgical region of the eye, and a surgical stage-based illumination module implemented in a non-transitory computer-readable storage medium and configured to perform operations including: determining, in real-time, a stage of the ophthalmic surgical procedure based on a detected trigger event, and outputting, in real-time, instructions to adjust the first illumination source or the second illumination source based on the stage of the ophthalmic surgical procedure.

In some aspects, the techniques described herein relate to an ophthalmic surgical system, wherein the detected trigger event includes actuation of an instrument for performing the stage of the ophthalmic surgical procedure.

In some aspects, the techniques described herein relate to an ophthalmic surgical system, wherein the surgical stage-based illumination module is configured to determine the detected trigger event based on image feed of the ophthalmic surgical procedure.

### Example Environment

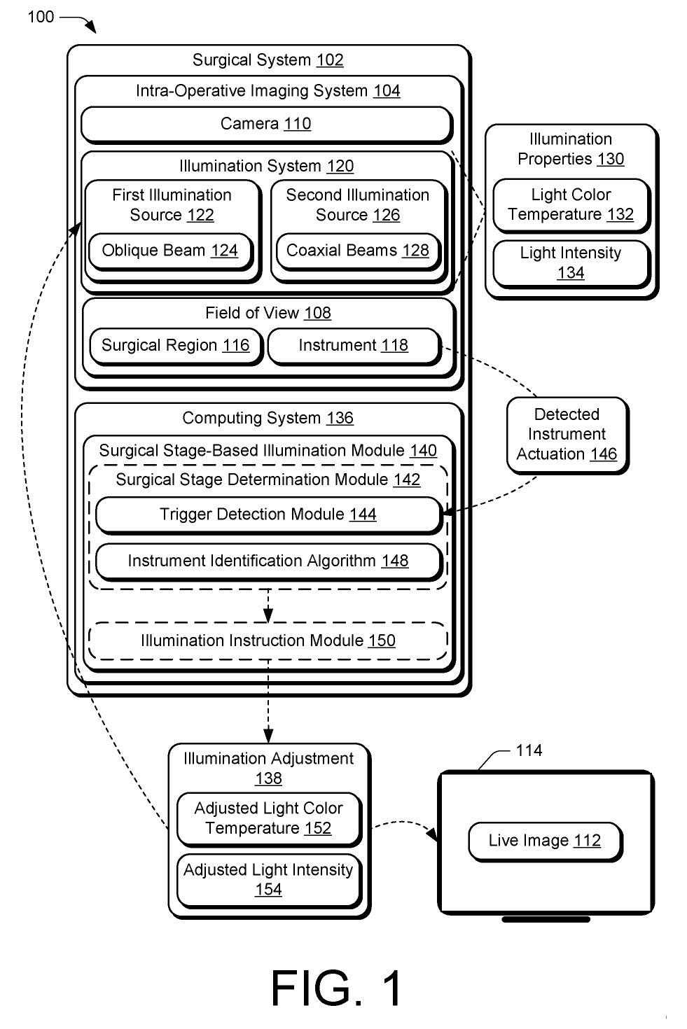

FIG. 1 is an illustration of an environment 100 in an example implementation that is operable to employ adjusting illumination sources based on stages of ophthalmic surgery, as described herein. The illustrated environment 100 includes a surgical system 102. The surgical system 102 may be a system configured to perform one or more ophthalmic surgical procedures, such as cataract surgery, glaucoma surgery, and the like, on an eye of a patient. The surgical system 102 includes an intra-operative imaging system 104 that obtains imaging data of a field of view 108 via a camera 110, sensor, or other device during an ophthalmic surgical procedure. By way of example, the intra-operative imaging system 104 may be a microscope, such as a digital microscope, a surgical microscope, or the like. In at least one implementation, a live image 112 including the imaging data from the ophthalmic surgical procedure may be displayed on a display device 114 substantially at the time of capture (e.g., without intentional delay) for a surgeon to view an increased level of detail of the field of view 108 during the ophthalmic surgical procedure. In at least one implementation, the live image 112 is a stereoscopic image.

The field of view 108 is representative of an area that is visible to the camera 110, e.g., through optical lenses, to obtain the image feed. In at least one implementation, such as the example indicated in FIG. 1, the field of view 108 includes at least a portion of a surgical region 116 of the patient (e.g., an eye of the patient, or a portion thereof) and at least a portion of an instrument 118 being used to perform the ophthalmic surgical procedure. As such, it is to be appreciated that during the ophthalmic surgical procedure, the intra-operative imaging system 104 is configured to obtain images (e.g., live images) depicting the procedure being performed on the patient.

The intra-operative imaging system 104 also includes an illumination system 120 to illuminate the surgical region 116 of the ophthalmic surgical procedure. The illumination system 120 may include a first illumination source 122, which in this example projects an oblique beam 124 to illuminate at least a portion of the surgical region 116. The oblique beam 124, for instance, is directed at an angled orientation (e.g., an angle other than 90 degrees), relative to a corneal plane of the eye 200 that is tangent to the vertex of the cornea 202, toward the surgical region 116 from the field of view 108 of the camera. The oblique beam 124 affects light phenomena at surfaces of the surgical region 116, such as reflection, refraction, and diffraction, depending on the nature of the surface and the medium the light is moving through. The illumination system 120 may also include a second illumination source 126, which in this example projects two or more coaxial beams 128 to illuminate at least a portion of the surgical region 116. The coaxial beams 128, in contrast to the oblique beam 124, for instance, are directed perpendicular (e.g., approximately 90 degrees relative to the corneal plane of the eye 200) to the surgical region 116 from the field of view 108 of the camera. The coaxial beams 128 propagate along a shared central line or path, resulting in a unified direction of light.

The first illumination source 122 and the second illumination source 126 have illumination properties 130, which are adjustable to configure light color temperature 132, light intensity 134, light illuminance, and other properties related to light. The light color temperature 132 refers to a level of light warmth, which determines whether the light appears “warm” (yellow/red) or “cool” (blue). The light intensity 134 refers to a measure of an amount of luminous flux emitted from a source within a given solid angle, expressed in units of candelas, or lumens per steradian. Intensity divided by the emitting area of the source is luminance is perceived as a “brightness” of the light. Therefore, for a given emitting area, increasing the intensity will increase the perceived brightness of the light. The light illuminance is a quantity defined as luminous flux per unit area, at the corneal plane, in units of lux, or lumens per square meter.

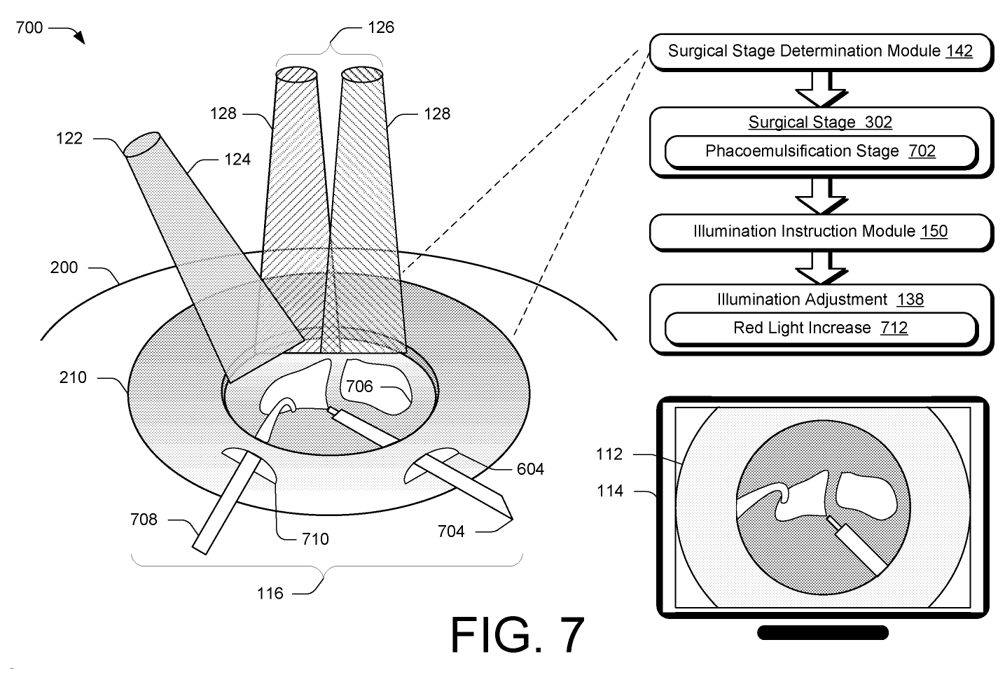

Different variations of light are optimal for different stages of ophthalmic surgical procedures. For example, illumination of a surgical region 116 using blue light is optimal for a surgeon during a capsulorhexis stage of a cataract surgery, in which the surgeon creates a circular opening in a lens capsule. This is because the blue light enhances contrast between different tissues, making the clear lens capsule stand out more distinctly against the surrounding tissues. This improved contrast helps the surgeon make precise cuts on the capsule. In contrast, illumination of the surgical region 116 using red light is optimal during a phacoemulsification stage of the cataract surgery, in which the surgeon breaks up and removes cataract material. This is because the red light highlights the red reflex, which is the red glow that appears when light reflects off the retina of the eye. The red reflex provides a visual contrast that helps the surgeon identify the boundaries of the cataract and surrounding structures, guiding precise instrument placement and movement. Additional examples of optimal lighting for stages of multiple ophthalmic surgical procedures are explained in relation to FIGS. 7-10.

To automatically adjust the illumination properties 130 based on a stage of the ophthalmic surgical procedure, the surgical system 102 includes a computing system 136, which is representative of one or more co-located or non-co-located systems that execute instructions to determine an illumination adjustment 138 using a surgical stage-based illumination module 140. A “module” may include a hardware and/or software system that operates to perform one or more functions, such as the functions that will be described below. For example, a module may include, or may be included in, a computer processor, a controller, or another logic-based device that performs operations based on instructions stored on a tangible and non-transitory computer readable storage medium, such as a computer memory. Alternatively, a module may include a hardwired device that performs operations based on hardwired logic of the device. The various modules shown in the attached figures, including FIG. 1, may represent the hardware that operates based on software or hardwired instructions, the software that directs hardware to perform the operations, or a combination thereof. The hardware may include electronic circuits that include and/or are connected to one or more logic-based devices, such as microprocessors, processors, controllers, and the like. As will be elaborated herein below, the illumination adjustment 138 causes adjustment related to actuation of the oblique beam 124, actuation of the coaxial beams 128, adjustment to the light color temperature 132, adjustment to the light intensity 134, and/or other adjustments to the illumination system 120.

In at least one implementation, the computing system 136 determines the illumination adjustment 138 using a surgical stage determination module 142 to first identify a current surgical stage of the ophthalmic surgical procedure. In at least one example, the surgical stage determination module 142 uses a trigger detection module 144 to determine the surgical stage based on a detected instrument actuation 146. For example, the trigger detection module 144 receives input data related to an instrument 118 is currently being used. The trigger detection module 144 is configured to analyze the input data to determine that the instrument 118 has been actuated and/or is still in use. For example, the trigger detection module 144 receives input data indicating that a phacoemulsification instrument has been actuated, and the trigger detection module 144 therefore determines that the phacoemulsification stage of the ophthalmic procedure is occurring.

In at least one additional or alternative implementation, the surgical stage determination module 142 uses an instrument identification algorithm 148 to identify the instrument 118 is currently used during the stage of ophthalmic surgery. The instrument identification algorithm 148, for instance, perform feature extraction (where edges, textures, and/or other key points are extracted from the image feed), object detection (e.g., where bounding region(s) of object(s) are located based on the extracted features), and classification (e.g., where the detected objects are classified based on trained categories). In at least one implementation, the instrument identification algorithm 148 involves a machine learning model trained using computer vision techniques to detect the targeted portion of the instrument 118 (e.g., a particular feature or features of the instrument 118) and/or a targeted anatomical feature. The machine learning model may be or may include a neural network (e.g., a convolutional neural network, a deep neural network, a recurrent neural network), a support vector machine, a transformer-based model, and/or a regression model (e.g., linear, polynomial, and/or logistic regression models), just to name a few. By way of example, the machine learning model may, through a training process, learn patterns in the image feed that are indicative of particular anatomical or instrument components. The underlying model(s) of the instrument identification algorithm 148 may be trained using different approaches, such as using supervised learning, unsupervised learning, semi-supervised learning, and/or reinforcement learning. Once trained, the instrument identification algorithm 148 is configured to use those one or more models to process the image feed in real-time to detect the targeted portion of the instrument 118 and the targeted anatomical feature.

It is to be appreciated that the instrument identification algorithm 148 may be configured as, or include, other types of models without departing from the spirit or scope of the described techniques. These types of models may be built or trained (or the model otherwise learned), respectively, using different algorithms and different data due, at least in part, to different architectures and/or learning paradigms.

In at least one variation, for instance, the instrument identification algorithm 148 receives input from the surgeon regarding which object(s) to recognize and localize in subsequent images. By way of example, rather than analyzing an entirety of the field of view 108, the instrument identification algorithm 148 tracks user-selected object(s) in subsequent image frames of the image feed. This may enable the instrument identification algorithm 148 to identify the user-selected object(s) in the subsequent image frames without specifically learning to identify and classify those object(s) through a training process.

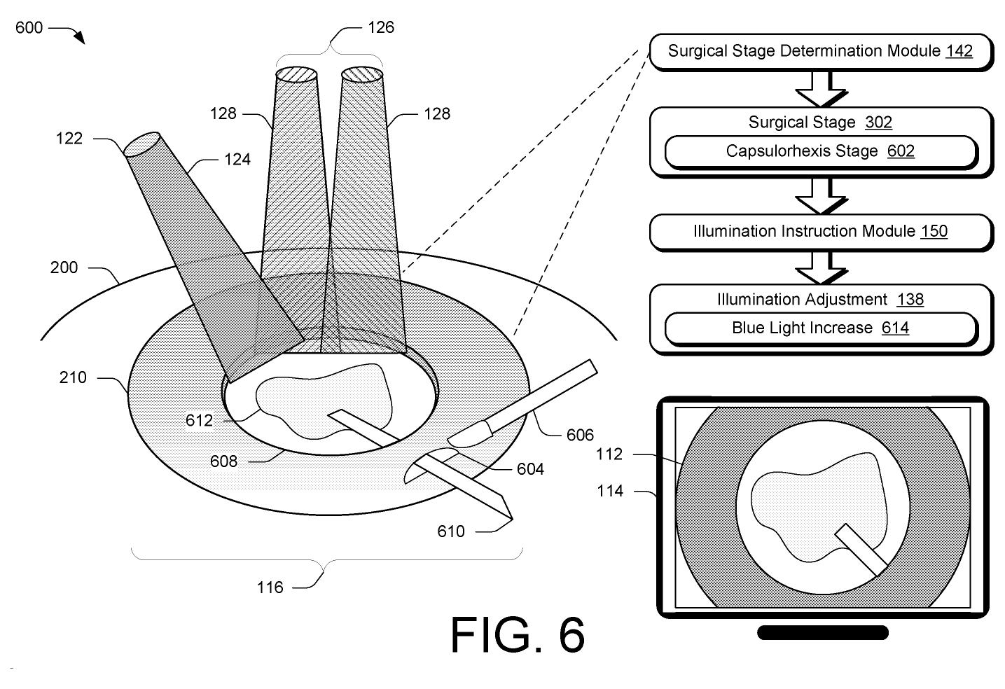

The surgical stage-based illumination module 140 also includes an illumination instruction module 150 that determines the illumination adjustment 138 based on the stage of the ophthalmic surgical procedure. For example, the illumination adjustment specifies an adjusted light color temperature 152 and/or an adjusted light intensity 154 for the first illumination source 122 and/or the second illumination source 126 of the illumination system 120. In an example, because the surgical stage determination module 142 determines that the current stage of the ophthalmic procedure is the phacoemulsification stage, the illumination instruction module 150 determines an illumination adjustment 138 to increase red content of light, including a preconfigured level of light color temperature 132 corresponding to the phacoemulsification stage. Additional examples of the illumination adjustment 138 for different stages of ophthalmic surgical procedures are provided below with respect to FIGS. 6-10.

In this way, the environment 100 provides an efficient system for adjusting illumination sources based on stages of ophthalmic surgery in real-time during an ophthalmic procedure. As a result, fewer illumination sources are controlled by the surgeon during the ophthalmic procedure, which decreases distractions, increases productivity, and increases occurrences of positive patient outcomes.

To facilitate the discussion of additional details of the real-time instrument position estimation guidance for ophthalmic surgery, an overview of anatomic features of the eye will now be provided.

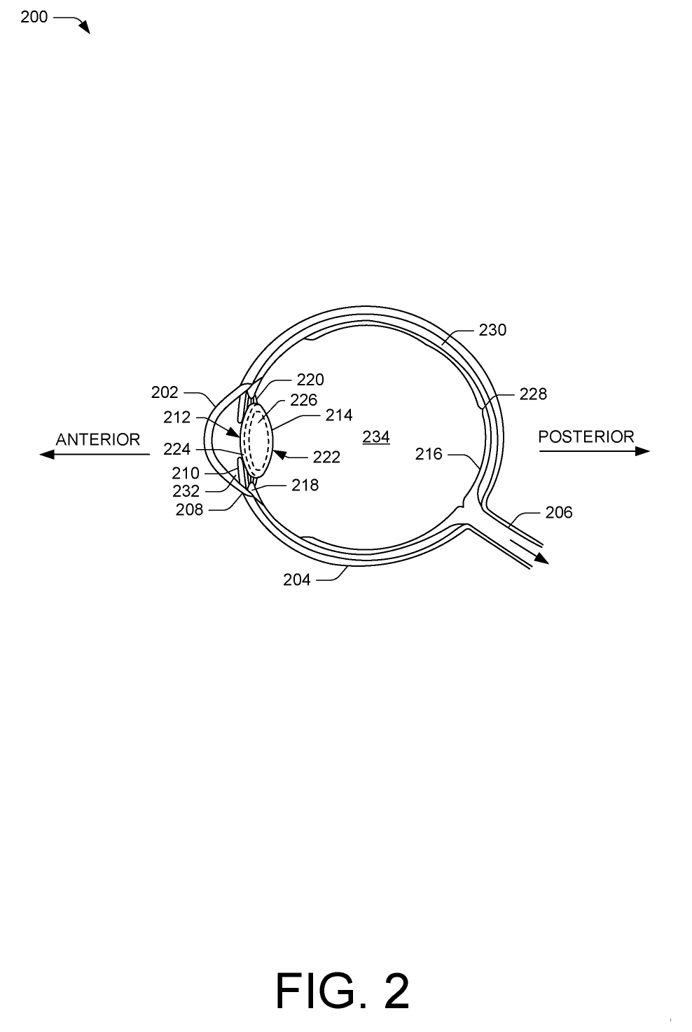

FIG. 2 depicts a cross-section of an eye 200. The eye 200 is a generally globular fused two-piece unit including a cornea 202 and sclera 204. The cornea 202 is a smaller transparent, dome-shaped front layer of the eye 200, whereas the sclera 204 is an opaque outer layer that provides structural support and extends from the cornea 202 to an optic nerve 206. The cornea 202 is connected to the sclera 204 at a limbus 208. The limbus 208, for instance, is a ring that provides a transition zone between the cornea 202 and the sclera 204. The limbus 208 is typically about 11-12 millimeters (mm) in width (e.g., horizontal diameter) and 10-11 mm in height (e.g., vertical diameter), appearing elliptical in shape. The cornea 202 comprises approximately one-sixth of the outer surface area of the eye 200, whereas the sclera 204 comprises the remaining five-sixths. Moreover, the cornea 202 protrudes beyond the globe of the sclera 204 due to having a different (e.g., smaller) radius of curvature than the sclera 204.

The eye 200 further includes an iris 210. The iris 210 is the colored part of the eye 200 and surrounds a pupil 212. The iris 210 controls the size of the pupil 212, which is the central opening that allows light to enter the eye. The pupil 212 adjusts the size of the pupil 212 in response to light intensity, such as by contracting to reduce the size of the pupil 212 in response to bright light and dilating to increase the size of the pupil 212 in response to dim light. The iris 210 and the pupil 212 are seen instead of the cornea 202 due to the transparency of the cornea 202.

The eye 200 further includes a lens 214 (e.g., a crystalline lens) located behind the iris 210 and the pupil 212. The lens 214 is a transparent, flexible, biconvex structure that focuses light onto retina 216, which is a thin layer of tissue lining the back of the eye. The lens 214 is attached to ciliary body 218 by suspensory ciliary ligaments 220 (e.g., Zonules of Zinn), which include fine transparent fibers. The ciliary body 218 adjusts the lens shape for near or distant vision. By way of example, the lens 214, by changing its shape, functions to change the focal distance of the eye 200 so that it can focus on objects at various distances, thus allowing a sharp real image of the object of interest to be formed on the retina 216. This adjustment of the lens 214 is known as accommodation and is similar to the focusing of a photographic camera via movement of its lenses.

The lens 214 has three main parts: a lens capsule 222, a lens epithelium 224, and lens fibers 226. The lens capsule 222 (e.g., capsular bag) forms the outermost layer of the lens 214, and the lens fibers 226 form the bulk of the interior of the lens 214. The cells of the lens epithelium 224, located between the lens capsule 222 and the outermost layer of the lens fibers 226, are found predominantly on the anterior side of the lens but extend posteriorly just beyond the equator. The lens capsule 222 is a smooth, transparent membrane that completely surrounds the lens 214. The lens capsule 222 is elastic and thus causes the lens 214 to assume a more globular shape when not under the tension of the lens capsule 222. The lens capsule 222 varies between approximately 2-28 micrometers in thickness, being thickest near the equator and thinnest near the posterior pole.

The eye 200 is an organ that reacts to light for several purposes. By way of example, as a conscious sense organ, the eye allows vision. Rod and cone cells in the retina 216 allow conscious light perception and vision, including color differentiation and the perception of depth. In addition, non-image-forming photosensitive ganglion cells in the retina 216 receive light signals that affect adjustment of the size of the pupil 212, regulation and suppression of the hormone melatonin, and entrainment of the body clock.

The retina 216 includes macula 228, which is a small, central area that provides detailed central vision. The optic nerve 206 transmits visual information from the retina 216 to the brain, e.g., via electrical signals. A layer called choroid 230 between the retina 216 and the sclera 204 is a vascular layer that provides circulation to the retina 216.

Thus, the eye 200 is made up of three layers that enclose three transparent structures. The outermost layer includes the cornea 202 and sclera 204. The middle layer includes the choroid 230, the ciliary body 218, and the iris 210. The innermost layer is the retina 216, which gets its circulation from the vessels of the choroid 230 as well as the retinal vessels, which can be seen within an ophthalmoscope. Within these coats are aqueous humor 232, vitreous humor 234, and the lens 214. The aqueous humor is a clear fluid that is contained in two areas: an anterior chamber between the cornea 202 and the iris 210 and the exposed area of the lens 214, and the posterior chamber, between the iris 210 and the lens 214. The vitreous humor 234 is a clear, gel-like substance that fills the space between the lens 214 and the retina 216 that helps maintain the shape of the eye 200 and allows light to pass through to the retina 216.

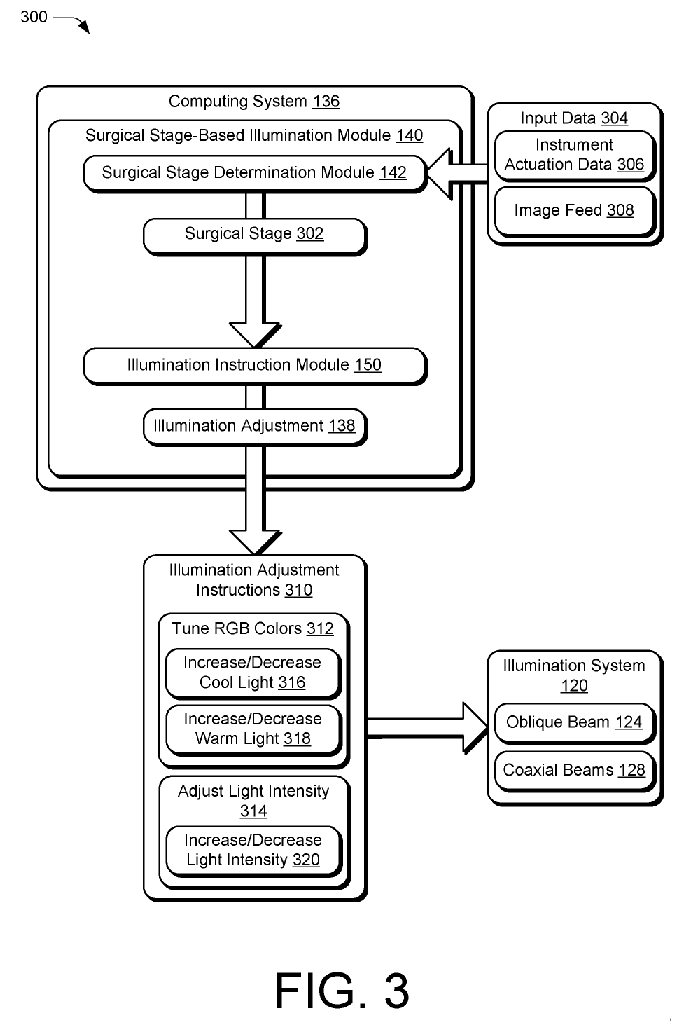

FIG. 3 depicts an example implementation 300 of the surgical stage-based illumination module 140 of FIG. 1 in greater detail. Where appropriate, reference will be made to components previously introduced in FIGS. 1 and 2.

In the implementation 300 shown in FIG. 3, the surgical stage-based illumination module 140 is implemented in a computing system 136 to perform operations related to determining an illumination adjustment 138 related to a surgical stage 302 of an ophthalmic surgical procedure. The surgical stage-based illumination module 140, for instance, may include a surgical stage determination module 142 that identifies the surgical stage 302. To do this, the surgical stage determination module 142 receives input data 304 including instrument actuation data 306, image feed 308, and/or other data related to stages of the ophthalmic surgical procedure. The instrument actuation data 306 relates to use of an instrument 118 for performing a particular stage or multiple stages of the ophthalmic surgical procedure and indicates whether the instrument 118 has been actuated (e.g., turned on) and/or is currently still in use.

The image feed 308 includes imaging data of a field of view 108 captured via a camera 110 or other device during the ophthalmic surgical procedure. In an example, the camera 110 is implemented into a microscope, such as a digital microscope, a surgical microscope, or the like. The field of view 108 is representative of an area that is visible to the camera 110, e.g., through optical lenses, to obtain the image feed. As such, the image feed 308 depicts the procedure being performed on the patient. The image feed 308 may depict an instrument 118 or other feature indicating a specific stage of the ophthalmic surgical procedure.

Based on the image feed 308 and/or other trigger, the surgical stage determination module 142 determines the surgical stage 302 of the ophthalmic surgical procedure. For example, the surgical stage determination module 142 may use a predetermined list of instruments that correspond to particular surgical stages. After analyzing the input data 304 including the instrument actuation data 306 and/or the image feed 308, the surgical stage determination module 142 matches a depicted instrument with the surgical stage 302. For example, the instrument actuation data 306 indicates that a phacoemulsification instrument has been actuated, and the surgical stage determination module 142 determines that the phacoemulsification instrument corresponds to the phacoemulsification stage.

In another implementation, the surgical stage determination module 142 leverages an instrument identification algorithm 148 to determine the surgical stage 302 based on the image feed 308. The instrument identification algorithm 148, for instance, is a machine learning model trained to identify the instrument in a frame of the image feed 308. To do this, the instrument identification algorithm 148 may perform feature extraction (where edges, textures, and/or other key points are extracted from the image feed), object detection (e.g., where bounding region(s) of object(s) are located based on the extracted features), and classification (e.g., where the detected objects are classified based on trained categories). The instrument identification algorithm 148 may be or may include a neural network (e.g., a convolutional neural network, a deep neural network, a recurrent neural network), a support vector machine, a transformer-based model, and/or a regression model (e.g., linear, polynomial, and/or logistic regression models), just to name a few. By way of example, the machine learning model may, through a training process, learn patterns in the image feed that are indicative of particular anatomical or instrument components.

In other implementations, the surgical stage determination module 142 leverages a machine learning model to identify features depicted in the image feed 308 that are used to identify the surgical stage 302, other than instruments. For example, the machine learning model may be trained to identify movements of a surgeon or changes in the surgical region 116 that indicate the surgical stage 302.

In some implementations, the input data 304 also includes a predetermined queue of planned surgical stages to aid the surgical stage determination module 142 in identifying the surgical stage 302. An example predetermined queue, for instance, may indicate a planned cataract surgery involving an incision stage, a capsulorhexis stage, a phacoemulsification stage, a lens implantation stage, and a closure stage. The surgical stage determination module 142 then analyzes the input data 304 to determine when one stage of the predetermined queue ends and another stage of the predetermined queue begins.

After the surgical stage determination module 142 determines the surgical stage 302, the surgical stage-based illumination module 140 uses an illumination instruction module 150 to determine an illumination adjustment 138. In an example, the illumination instruction module 150 uses a predetermined list of instruments that correspond to different illumination adjustments. For example, the phacoemulsification stage corresponds to an increase in red light. In some example implementations, the illumination adjustments are customizable by a surgeon or other user to preconfigure the illumination adjustments based on personal preferences for different surgical stages.

Based on the illumination adjustment 138, the illumination instruction module 150 outputs illumination adjustment instructions 310 indicating whether to tune RGB colors 312, adjust light intensity 314, actuate the oblique beam 124 on or off, actuate the coaxial beams 128 on or off, or other instructions related to the illumination system 120. The instructions to tune RGB colors 312, for instance, include an increase or decrease in cool light 316 or an increase of decrease in warm light 318. The instructions to adjust light intensity 314 include an increase or decrease to light intensity 320. In this example, the illumination adjustment instructions 310 are output to inform the illumination system 120 to adjust the oblique beam 124 and/or the coaxial beams 128.

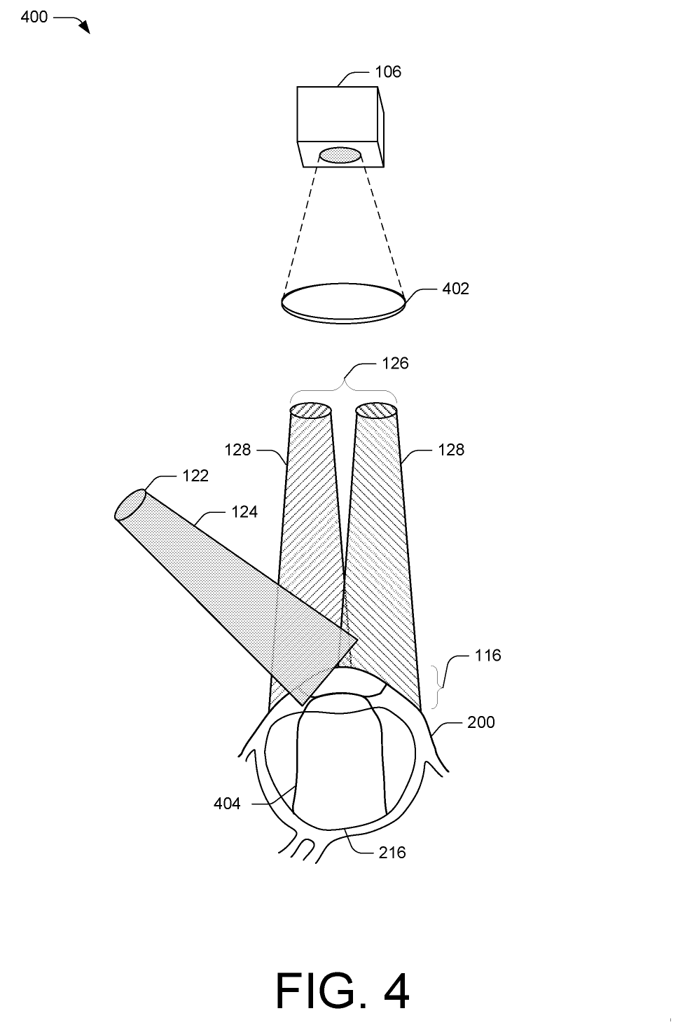

FIG. 4 depicts a non-limiting example 400 of illumination sources used in the context of an ophthalmic surgical procedure. Specifically, the non-limiting example 400 shows an example illumination system 120 including a first illumination source 122 and a second illumination source 126. However, other implementations may use other numbers of illuminations sources.

As illustrated, the first illumination source 122 in this example is an LED light engine that projects an oblique beam 124 to illuminate at least a portion of a surgical region 116 of an eye for an ophthalmic surgical procedure. The oblique beam 124, for instance, is directed at an angled orientation (e.g., an angle other than 90 degrees) toward the surgical region 116 from the field of view 108 of a camera 110. The oblique beam 124 affects light phenomena at surfaces of the surgical region 116, such as reflection, refraction, and diffraction, depending on the nature of the surface and the medium the light is moving through. For example, the oblique beam 124 increases contrast of the surgical region 116 of the eye 200 when viewed from the field of view 108 of the camera 110, which is facing the cornea 202 of the eye 200.

The second illumination source 126 in this example includes two or more LED light engines that project two or more coaxial beams 128 to illuminate at least a portion of the surgical region 116. The coaxial beams 128, in contrast to the oblique beam 124, for instance, are directed perpendicular (e.g., approximately 90 degrees) to the surgical region 116 from the field of view 108 of the camera 110, relative to a corneal plane of the eye. The coaxial beams 128 propagate along a shared central line or path, resulting in a unified direction of light. In comparison, the oblique beam 124 can be at an angle of between 4 and 60 degrees relative to an optical axis of the eye 200 that is perpendicular to the corneal plane.

In this example, the surgical region 116 is viewed via an objective lens 402 via the camera 110. For instance, the objective lens 402 is part of the intra-operative imaging system 104 described with respect to FIG. 1, including the surgical microscope or other visual aid device used to magnify and enhance the surgeon’s view of internal structures of the eye 200. Specifically, the objective lens provides clear, high-resolution, and magnified images of an anterior segment of the eye 200, including the cornea 202, the lens 214, and surrounding tissues. This allows the surgeon to see fine details related to performing delicate tasks for various ophthalmic surgical procedures, such as cataract surgery, including capsulorhexis and phacoemulsification stages. Reflected light 404 results from the oblique beam 124 and the coaxial beams 128 and is reflected off regions of the eye 200, including the retina 216. The reflected light 404 further aids in presenting the details of the eye 200 related to the cataract surgery.

Although the non-limiting example 400 is described with respect to cataract surgery, it is to be appreciated that the described techniques are applicable to other types of ophthalmic surgeries or techniques. Additionally, although the non-limiting example 400 is described is described as including a first illumination source 122 projecting an oblique beam 124 and a second illumination source 126 projecting coaxial beams 128, it is to be appreciated that the described techniques are applicable to other configurations of illumination systems that include other illumination sources and/or project other type of beams of light.

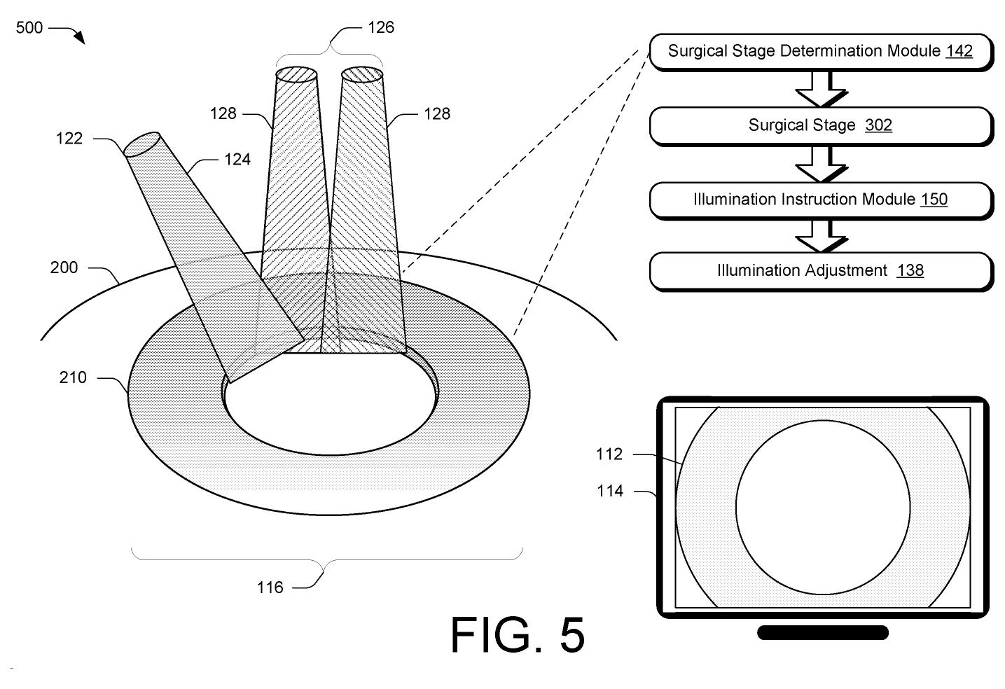

FIG. 5 depicts a non-limiting illustrative example overview 500 of adjusting illumination sources based on stages of an ophthalmic surgical procedure. It is to be appreciated that the non-limiting illustrative example overview 500 is a simplified, exaggerated view to provide visualization of the values and features discussed herein. Where appropriate, reference is made to components and features previously introduced with respect to FIGS. 1-4.

As illustrated, an eye 200 is prepared for an ophthalmic surgical procedure. To view the surgical region 116 of the eye 200 with clarity, an intra-operative imaging system 104 obtains imaging data of the surgical region 116 via a camera 110 or other device. For example, the intra-operative imaging system 104 may be a microscope, such as a digital microscope, a surgical microscope, or the like. As depicted in this example, a live image 112 including the imaging data from the ophthalmic surgical procedure may be displayed on a display device 114 substantially at the time of capture (e.g., without intentional delay) for a surgeon to view an increased level of detail of the surgical region 116 during the ophthalmic surgical procedure.

To illuminate the surgical region 116 and to provide a live image 112 with a high level of visibility, an illumination system 120 illuminates the surgical region 116 of the ophthalmic surgical procedure. The illumination system 120 may include a first illumination source 122, which in this example projects an oblique beam 124 to illuminate at least a portion of the surgical region 116. The oblique beam 124, for instance, is directed at an angled orientation (e.g., an angle other than 90 degrees) toward the surgical region 116 from the field of view 108 of the camera. The oblique beam 124 affects light phenomena at surfaces of the surgical region 116, such as reflection, refraction, and diffraction, depending on the nature of the surface and the medium the light is moving through. The illumination system 120 in this example also includes a second illumination source 126, which in this example projects two or more coaxial beams 128 to illuminate at least a portion of the surgical region 116. The coaxial beams 128, in contrast to the oblique beam 124, for instance, are directed perpendicular (e.g., approximately 90 degrees) to the surgical region 116 from the field of view 108 of the camera, relative to a corneal plane of the eye. The coaxial beams 128 propagate along a shared central line or path, resulting in a unified direction of light.

As described with respect to FIG. 3., the first illumination source 122 and the second illumination source 126 have illumination properties 130, which are adjustable to configure light color temperature 132, light intensity 134, and other properties related to light. The light color temperature 132 refers to a color temperature of light, which determines whether the light appears “warm” (yellow/red) or “cool” (blue). The light intensity 134 refers to a measure of an amount of light energy emitted from a source in a given direction, which is perceived as a “brightness” of the light.

Different variations of light are optimal for different stages of ophthalmic surgical procedures. To automatically adjust the illumination properties 130 based on a stage of the ophthalmic surgical procedure, the surgical stage-based illumination module 140 uses a surgical stage determination module 142 to determine a surgical stage 302 of the ophthalmic surgical procedure. For example, the surgical stage determination module 142 may receive instrument actuation data 306 and determine the surgical stage 302 based on which instrument(s) are actuated. Additionally or alternatively, the surgical stage determination module 142 may receive image feed 308 and use an instrument identification algorithm 148 to identify instruments and/or surgical processes captured in the image feed 308 that indicate the surgical stage 302. The surgical stage determination module 142 is described in further detail with respect to FIGS. 6-9.

After the surgical stage determination module 142 determines the surgical stage 302, the surgical stage-based illumination module 140 uses an illumination instruction module 150 to determine an illumination adjustment 138 for the first illumination source 122 and/or the second illumination source 126. For example, the illumination adjustment specifies an adjusted light color temperature 152 and/or an adjusted light intensity 154 for the first illumination source 122 and/or the second illumination source 126 of the illumination system 120.

In some example implementations, the illumination instruction module 150 may also determine a zoom adjustment for the live image 112 of the image feed 308 based on the surgical stage 302. For instance, a zoomed-out view is optimal for some surgical stages, while a zoomed-in view is optimal for other surgical stages, based on individual surgeon preferences. Based on the surgical stage 302, for instance, the illumination instruction module 150 determines, based on predetermined preferences related to image zoom, a zoom adjustment related to the camera 110, the live image 112, and/or the image feed 308 that corresponds to the surgical stage 302.

Having discussed example details of the techniques for adjusting illumination sources based on stages of ophthalmic surgery, consider now an example to illustrate usage of the techniques related to specific surgical procedures, including examples of cataract surgery and glaucoma surgery.

### Example Applications

FIG. 6 depicts a non-limiting illustrative example 600 of adjusting illumination sources based on a capsulorhexis stage 602 of cataract surgery. Components that function similarly are numbered the same as those previously introduced in FIGS. 1-5. The example 600 is a continuation of the non-limiting illustrative example overview 500 shown with respect to FIG. 5.

As shown, the capsulorhexis stage 602 of cataract surgery is performed. During the capsulorhexis stage 602, a corneal incision 604 is made into the lens capsule using an incision instrument 606 to remove a cataract 608. Additionally, in some examples a dye injector instrument 610 is used to inject dye 612 into an anterior chamber of the eye 200 to increase contrast for viewing the capsulorhexis stage 602.

As discussed with respect to FIGS. 4 and 5, a first illumination source 122 projects an oblique beam 124 at the surgical region 116, and a second illumination source 126 projects coaxial beams 128 at the surgical region 116. The first illumination source 122 and the second illumination source 126 are further configurable to adjust light color temperature 132 and light intensity 134 of the oblique beam 124 and the coaxial beams 128. Because different stages of ophthalmic surgical procedures involve different optimal levels of light warmth of color and light intensity, a surgical stage determination module 142 is configured to identify the surgical stage 302, which is used to determine corresponding lighting adjustment.

In this example, the surgical stage determination module 142 identifies the incision instrument 606 and/or the dye injector instrument 610 or other instrument associated with the capsulorhexis stage 602. For instance, in some examples, the surgical stage determination module 142 may receive instrument actuation data 306 indicating that the incision instrument 606 and/or the dye injector instrument 610 has been actuated. Additionally or alternatively, the surgical stage determination module 142 may receive image feed 308 and use an instrument identification algorithm 148 to identify the incision instrument 606 and/or dye injector instrument 610 in the live image 112, indicating the capsulorhexis stage 602.

During the capsulorhexis stage 602, a blue light increase 614 is optimal because the blue light enhances contrast between different tissues, making the clear lens capsule stand out more distinctly against the surrounding tissues. This improved contrast helps the surgeon make precise cuts on the capsule. For this reason, the illumination instruction module 150 determines an illumination adjustment 138 indicating the blue light increase 614, which is an illumination adjustment preassigned to the capsulorhexis stage 602. As illustrated, the first illumination source 122 and the second illumination source 126 are adjusted based on instructions output by the illumination adjustment 138. Accordingly, the blue light increase 614 is applied to the oblique beam 124 and the coaxial beams 128. This results in an increase in contrast, which is reflected in the live image 112 displayed on the display device 114.

In this way, the example 600 demonstrates how the adjusting illumination sources based on stages of ophthalmic surgery techniques can be applied to the capsulorhexis stage 602 of a cataract surgical procedure to improve the precision and outcomes of the procedure. For instance, increasing visibility of the live image 112 using the blue light increase 614 enhances contrast, potentially reducing the risk of complications during the procedure.

FIG. 7 depicts a non-limiting illustrative example 700 of adjusting illumination sources based on a phacoemulsification stage 702 of cataract surgery. Components that function similarly are numbered the same as those previously introduced in FIGS. 1-6. The example 700 is a continuation of the example 600 shown with respect to FIG. 6.

As shown, the phacoemulsification stage 702 of cataract surgery is performed. During the phacoemulsification stage 702, a phacoemulsification instrument 704 or Constellation instrument is inserted through the corneal incision 604 to break up the cataract into fragments 706, which are removed via aspiration using a suction functionality of the phacoemulsification instrument 704. Additional tools 708, including forceps, scissors, or needle holders, may also be used to make incisions, manipulate tissues, and/or suture incisions. These additional tools 708 may be inserted using an additional corneal incision 710.

As discussed with respect to FIGS. 4 and 5, a first illumination source 122 projects an oblique beam 124 at the surgical region 116, and a second illumination source 126 projects coaxial beams 128 at the surgical region 116. The first illumination source 122 and the second illumination source 126 are further configurable to adjust light color temperature 132 and light intensity 134 of the oblique beam 124 and the coaxial beams 128. Because different stages of ophthalmic surgical procedures involve different optimal levels of light warmth of color and light intensity, a surgical stage determination module 142 is configured to identify the surgical stage 302, which is used to determine corresponding lighting adjustment.

In this example, the surgical stage determination module 142 identifies the phacoemulsification instrument 704, the Constellation instrument, or other instrument associated with the phacoemulsification stage 702. For instance, in some examples, the surgical stage determination module 142 may receive instrument actuation data 306 indicating that the phacoemulsification instrument 704 has been actuated. Additionally or alternatively, the surgical stage determination module 142 may receive image feed 308 and use an instrument identification algorithm 148 to identify the phacoemulsification instrument 704 in the live image 112, indicating the phacoemulsification stage 702.

During the phacoemulsification stage 702, a red light increase 712 is optimal because the red light highlights the red reflex, which is the red glow that appears when light reflects off the retina at the back of the eye 200. The red reflex, for instance, refers to light that is coaxial illumination passed through the lens of the eye 200, focused down onto a spot on the retina 216, diffusively reflected off the retina 216, passed back out of and collimated by the lens 214 of the eye 200, and travels into the camera 110 of the microscope, where it is viewed by a surgeon. The red reflex provides backlighting of the anterior chamber, such that tissue structures within the anterior chamber are silhouetted by the backlit red light of the red reflex. The brighter the red reflex light, the stronger the visual contrast of the silhouetted structures in the foreground, which can lead to better surgical outcomes. The retina preferentially reflects the red light at a higher reflection efficiency than yellow, green or blue light. Therefore, if the color of the white coaxial light is adjusted to have greater red light content (lower color temperature), the brightness of the red reflex light will be higher. The red reflex provides a visual contrast that helps the surgeon identify the boundaries of the cataract and surrounding structures, guiding precise instrument placement and movement.

For this reason, the illumination instruction module 150 determines an illumination adjustment 138 indicating the red light increase 712, which is an illumination adjustment preassigned to the phacoemulsification stage 702 in this example. As illustrated, the first illumination source 122 and the second illumination source 126 are adjusted based on instructions output by the illumination adjustment 138. Accordingly, the red light increase 712 is applied to the coaxial beams 128. This results in an increase in contrast using the red reflex, which is reflected in the live image 112 displayed on the display device 114.

In this way, the example 700 demonstrates how the adjusting illumination sources based on stages of ophthalmic surgery techniques can be applied to the phacoemulsification stage 702 of a cataract surgical procedure to improve the precision and outcomes of the procedure. For instance, increasing visibility of the live image 112 using the red light increase 712 enhances contrast using the red reflex, potentially reducing the risk of complications during the procedure.

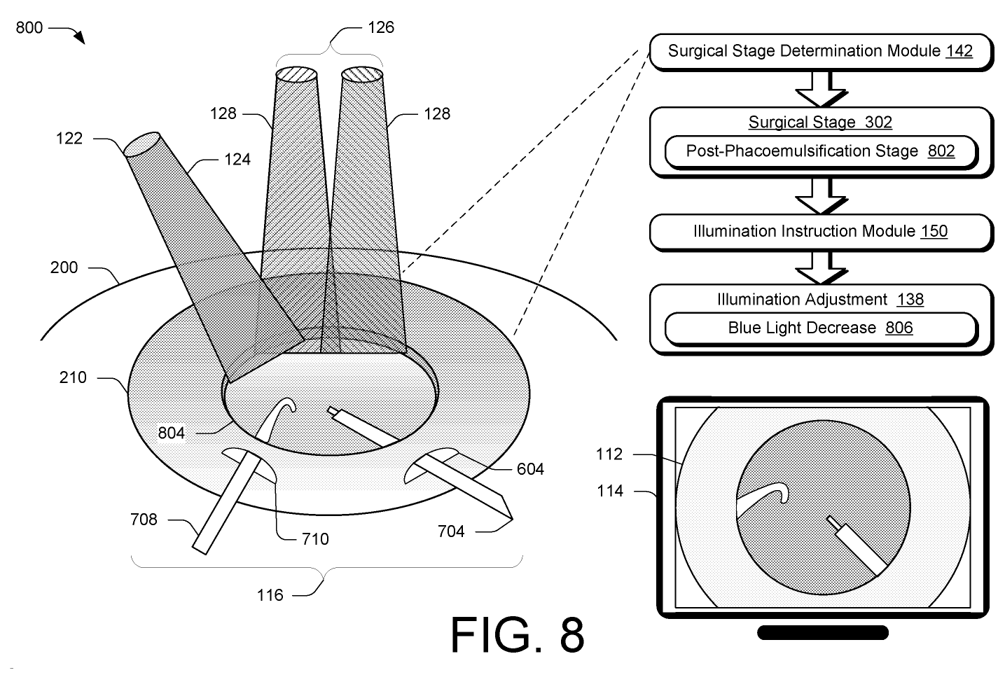

FIG. 8 depicts a non-limiting illustrative example 800 of adjusting illumination sources based on a post-phacoemulsification stage 802 of cataract surgery. Components that function similarly are numbered the same as those previously introduced in FIGS.

1-7. The example 800 is a continuation of the example 700 shown with respect to FIG. 7.

As shown, the post-phacoemulsification stage 802 of cataract surgery is performed. Following the phacoemulsification stage 702, for instance, the cataract is removed, leaving the eye 200 without a natural lens, as illustrated by the exposed retina 804.

As discussed with respect to FIGS. 4 and 5, a first illumination source 122 projects an oblique beam 124 at the surgical region 116, and a second illumination source 126 projects coaxial beams 128 at the surgical region 116. The first illumination source 122 and the second illumination source 126 are further configurable to adjust light color temperature 132 and light intensity 134 of the oblique beam 124 and the coaxial beams 128. Because different stages of ophthalmic surgical procedures involve different optimal levels of light warmth of color and light intensity, a surgical stage determination module 142 is configured to identify the surgical stage 302, which is used to determine corresponding lighting adjustment.

In this example, the surgical stage determination module 142 identifies the phacoemulsification instrument 704 or other instrument associated with the phacoemulsification stage 702 and concludes that the cataract has been removed. For instance, in some examples, the surgical stage determination module 142 may receive instrument actuation data 306 indicating that the phacoemulsification instrument 704 is at a predetermined stage of use, is near completion, or has ended. Additionally or alternatively, the surgical stage determination module 142 may receive image feed 308 and use an instrument identification algorithm 148 to identify the phacoemulsification instrument 704 in the live image 112, indicating the phacoemulsification stage 702 is at a predetermined stage of use, is near completion, or has ended.

During the post-phacoemulsification stage 802, a blue light decrease 806 is optimal because blue light is harmful to the exposed retina 804 of the patient. For instance, the natural lens, which has been removed, helps block blue light, which is a part of the visible spectrum that can be damaging in large amounts. Without a lens, the retina 216 is exposed to more intense light than usual. The blue light, which has higher energy and penetrates deeper into the eye 200, could cause discomfort or damage to retinal cells if exposure is prolonged. Additionally, the blue light is associated with a higher risk of phototoxicity, or light-induced damage to retinal cells.

For this reason, the illumination instruction module 150 determines an illumination adjustment 138 indicating the blue light decrease 806, which is an illumination adjustment preassigned to the post-phacoemulsification stage 802. As illustrated, the first illumination source 122 and the second illumination source 126 are adjusted based on instructions output by the illumination adjustment 138. Accordingly, the blue light decrease 806 is applied to the oblique beam 124 and the coaxial beams 128. This results in a decrease in blue light entering the exposed retina 804, which is reflected in the live image 112 displayed on the display device 114.

In this way, the example 800 demonstrates how the adjusting illumination sources based on stages of ophthalmic surgery techniques can be applied to the post-phacoemulsification stage 802 of a cataract surgical procedure to improve the outcomes of the procedure. For instance, decreasing the blue light entering the patient’s retina potentially reduces the risk of complications resulting from the procedure.

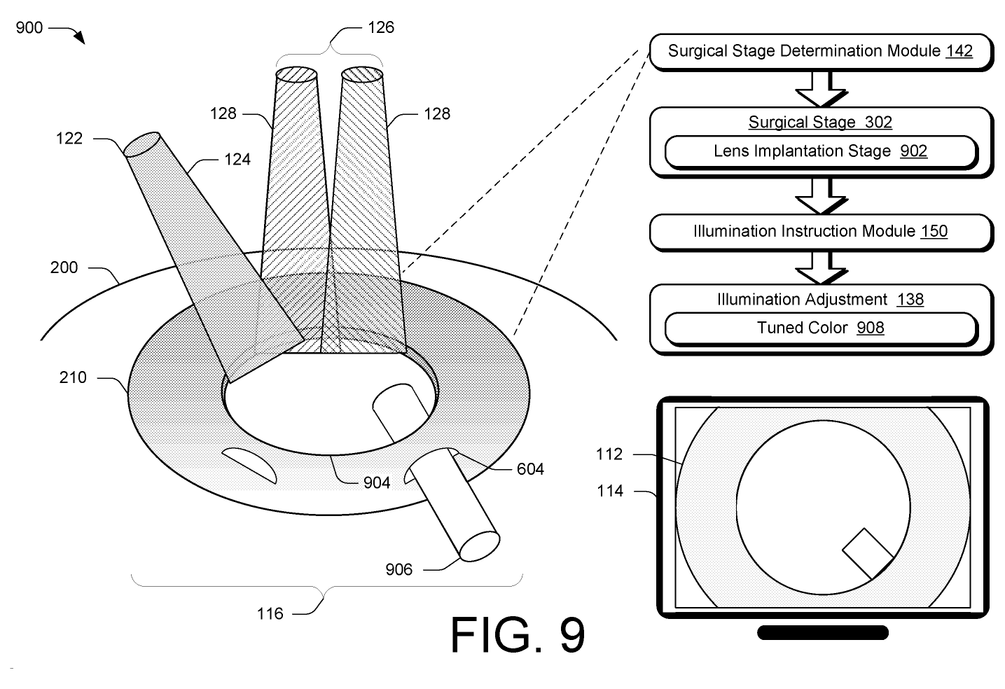

FIG. 9 depicts a non-limiting illustrative example 900 of adjusting illumination sources based on a lens implantation stage 902 of cataract surgery. Components that function similarly are numbered the same as those previously introduced in FIGS. 1-8. The example 900 is a continuation of the example 800 shown with respect to FIG. 8.

As shown, the lens implantation stage 902 of cataract surgery is performed. During the lens implantation stage 902, an intraocular lens 904 is implanted to replace the removed natural lens using a lens implantation instrument 906. The intraocular lens 904 is made of a flexible material, such as silicone or acrylic, allowing it to be folded for easy insertion through the corneal incision 604 using the lens implantation instrument 906. In some examples, the lens implantation instrument 906 is an injector that helps guide the intraocular lens 904 precisely into the lens capsular bag of the eye 200, a thin, transparent membrane that originally held the natural lens. Once inside the eye 200, the intraocular lens 904 unfolds and is carefully positioned in the capsular bag to align with the eye’s natural light pathway. The intraocular lens 904 features small “haptics,” or arms, that help secure it in place within the capsular bag, preventing shifting.

The intraocular lens 904, for instance, may be positioned using Purkinje imaging, which involves capturing and analyzing the reflections of light from different surfaces within the eye 200. Purkinje images are created by light reflecting off various ocular interfaces, including Purkinje Image I, which is the reflection from the outer surface of the cornea 202; Purkinje Image II, which is the reflection from the inner surface of the cornea 202, Purkinje Image III, which is the reflection from the outer (anterior) surface of the lens, and Purkinje Image IV, which is the reflection from the inner (posterior) surface of the lens.

As discussed with respect to FIGS. 4 and 5, a first illumination source 122 projects an oblique beam 124 at the surgical region 116, and a second illumination source 126 projects coaxial beams 128 at the surgical region 116. The first illumination source 122 and the second illumination source 126 are further configurable to adjust light color temperature 132 and light intensity 134 of the oblique beam 124 and the coaxial beams 128. Because different stages of ophthalmic surgical procedures involve different optimal levels of light warmth of color and light intensity, a surgical stage determination module 142 is configured to identify the surgical stage 302, which is used to determine corresponding lighting adjustment.

In this example, the surgical stage determination module 142 identifies the lens implantation instrument 906 or other instrument associated with the lens implantation stage 902. For instance, in some examples, the surgical stage determination module 142 may receive instrument actuation data 306 indicating that the lens implantation instrument 906 has been actuated. Additionally or alternatively, the surgical stage determination module 142 may receive image feed 308 and use an instrument identification algorithm 148 to identify the lens implantation instrument 906 in the live image 112, indicating the lens implantation stage 902 has commenced.

During the lens implantation stage 902, a tuned color 908 is optimal based on individual surgeon preferences. For instance, the illumination instruction module 150 determines the tuned color 908 based on a predetermined preference for illumination to identify the Purkinje during the lens implantation stage 902. In an example, the tuned color 908 results in increased contrast to the surgeon may successfully implant the intraocular lens 904.

For this reason, the illumination instruction module 150 determines an illumination adjustment 138 indicating the tuned color 908, which is an illumination adjustment preassigned to the lens implantation stage 902. As illustrated, the first illumination source 122 and the second illumination source 126 are adjusted based on instructions output by the illumination adjustment 138. Accordingly, the tuned color 908 is applied to the oblique beam 124 and the coaxial beams 128.

In this way, the example 900 demonstrates how the adjusting illumination sources based on stages of ophthalmic surgery techniques can be applied to the lens implantation stage 902 of a cataract surgical procedure to improve the outcomes of the procedure. For instance, adjusting the illumination sources based on surgeon preferences for the lens implantation stage 902 reduces the risk of complications resulting from the procedure.

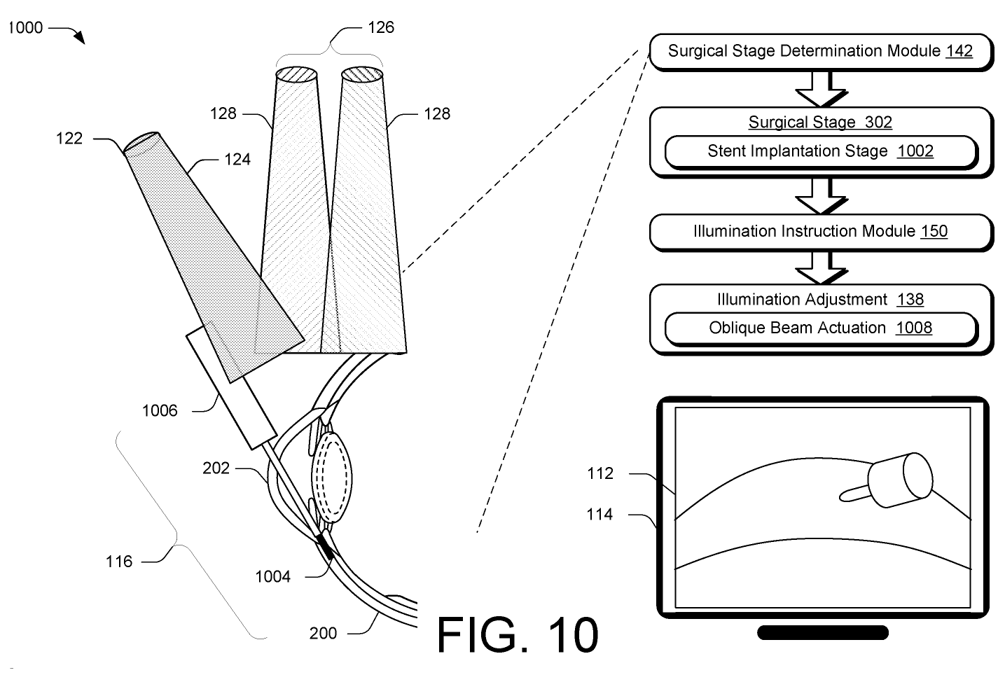

FIG. 10 depicts a non-limiting illustrative example 1000 of adjusting illumination sources based on a stent implantation stage 1002 of glaucoma surgery. Components that function similarly are numbered the same as those previously introduced in FIGS. 1-9.

As shown, the stent implantation stage 1002 of glaucoma surgery is performed. During the stent implantation stage 1002, a stent 1004 is inserted into a trabecular meshwork, which it a natural drainage pathway of the eye. This allows aqueous humor, which is the eye’s natural fluid, to bypass areas of resistance and flow more freely out of the eye, via the stent 1004. To do so, a small incision is made in the cornea 202 to access the eye’s trabecular meshwork. Using a stent implantation instrument 1006, the stent 1004 is inserted through the incision and positioned in place within the trabecular meshwork.

As discussed with respect to FIGS. 4 and 5, a first illumination source 122 projects an oblique beam 124 at the surgical region 116, and a second illumination source 126 projects coaxial beams 128 at the surgical region 116. The first illumination source 122 and the second illumination source 126 are further configurable to adjust light color temperature 132 and light intensity 134 of the oblique beam 124 and the coaxial beams 128. Because different stages of ophthalmic surgical procedures involve different optimal levels of light warmth of color and light intensity, a surgical stage determination module 142 is configured to identify the surgical stage 302, which is used to determine corresponding lighting adjustment.

In this example, the surgical stage determination module 142 identifies the stent implantation instrument 1006 or other instrument associated with the stent implantation stage 1002. For instance, in some examples, the surgical stage determination module 142 may receive instrument actuation data 306 indicating that the stent implantation instrument 1006 has been actuated. Additionally or alternatively, the surgical stage determination module 142 may receive image feed 308 and use an instrument identification algorithm 148 to identify the stent implantation instrument 1006 in the live image 112, indicating the stent implantation stage 1002 has commenced.

During the stent implantation stage 1002, an oblique beam actuation 1008 is optimal for illuminating the surgical region 116, based on individual surgeon preferences. This is because the oblique beam 124 is positioned to align with the diagonal insertion angle of the stent 1004, so the surgeon may clearly view operation of the stent implantation instrument 1006 and correctly insert the stent 1004.

For this reason, the illumination instruction module 150 determines an illumination adjustment 138 indicating the oblique beam actuation 1008, which is an illumination adjustment preassigned to the stent implantation stage 1002. In some examples, this involves tuning the first illumination source 122 and/or the second illumination source 126 so that the oblique beam 124 is brighter, has increased light intensity, or has different illumination characteristics compared to the coaxial beams 128. As illustrated, the first illumination source 122 and the second illumination source 126 are adjusted based on instructions output by the illumination adjustment 138. Accordingly, the tuned the oblique beam 124 is actuated to illuminate the surgical region 116 of the glaucoma surgical procedure.

In this way, the example 1000 demonstrates how the adjusting illumination sources based on stages of ophthalmic surgery techniques can be applied to the stent implantation stage 1002 of a glaucoma surgical procedure to improve the outcomes of the procedure. For instance, actuating the oblique beam for the stent implantation stage 1002 reduces the risk of complications resulting from the procedure.

Having discussed example details of the techniques for adjusting illumination sources based on stages of ophthalmic surgery, consider now an example procedure to illustrate additional aspects of the techniques.

### Example Procedure

This section describes an example procedure for adjusting illumination sources based on stages of ophthalmic surgery in one or more implementations. Aspects of the procedure may be implemented in hardware, firmware, or software, or a combination thereof. The procedure is shown as a set of blocks that specify operations performed by one or more devices and are not necessarily limited to the orders shown for performing the operations by the respective blocks. In at least some implementations, at least a portion of the procedure is performed by a suitably configured device, such as the computing system 136 of FIG. 1, by executing instructions stored in a non-transitory computer-readable storage medium.

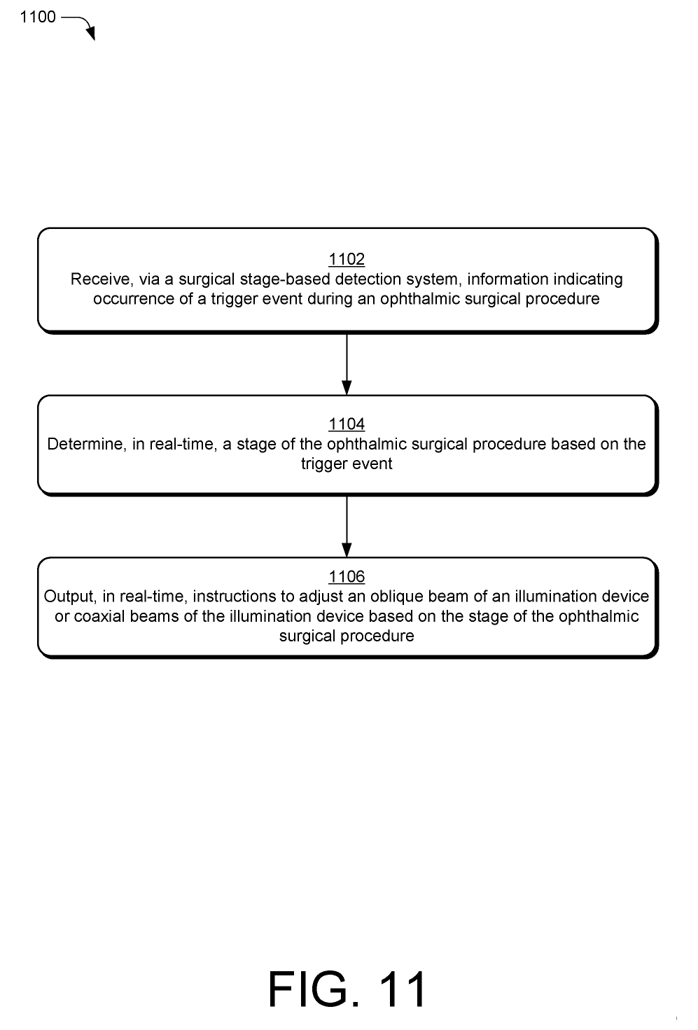

FIG. 11 depicts an example procedure 1100 in which adjusting illumination sources based on stages of ophthalmic surgery is performed.

Information indicating occurrence of a trigger event is received during an ophthalmic surgical procedure (block 1102). By way of example, the detected trigger event includes actuation of an instrument 118 associated with performing the stage of the ophthalmic surgical procedure. Additionally or alternatively, some examples further comprise determining the detected trigger event based on the image feed 308 of the ophthalmic surgical procedure. For example, an instrument identification algorithm 148 is used to identify an instrument associated with performing the stage of the ophthalmic surgical procedure in the image feed 308.

An identification of a stage of the ophthalmic surgical procedure is made in real-time based on the trigger event (block 1104). In some example implementations, information is received that indicates a relation between an instrument 118 and the stage of the ophthalmic surgical procedure. The stage of the ophthalmic surgical procedure, for instance, is identified based on the instrument 118 associated with the stage of the ophthalmic surgical procedure.

Instructions are output in real-time to adjust an oblique beam 124 of an illumination device or coaxial beams 128 of the illumination device based on the stage of the ophthalmic surgical procedure (block 1106). For example, the oblique beam 124 is directed at a diagonal angle toward a cornea 202 of an eye 200, and the coaxial beams 128 are directed at a perpendicular angle toward the cornea 202 of the eye 200, relative to a corneal plane of the eye 200. Some examples further comprise adjusting a light color temperature 132 of the oblique beam 124 or the coaxial beams 128 by tuning colors of the oblique beam 124 or the coaxial beams 128 based on the stage of the ophthalmic surgical procedure. Additionally, some examples further comprise tuning an intensity of the oblique beam 124 or the coaxial beams 128 source based on the stage of the ophthalmic surgical procedure.

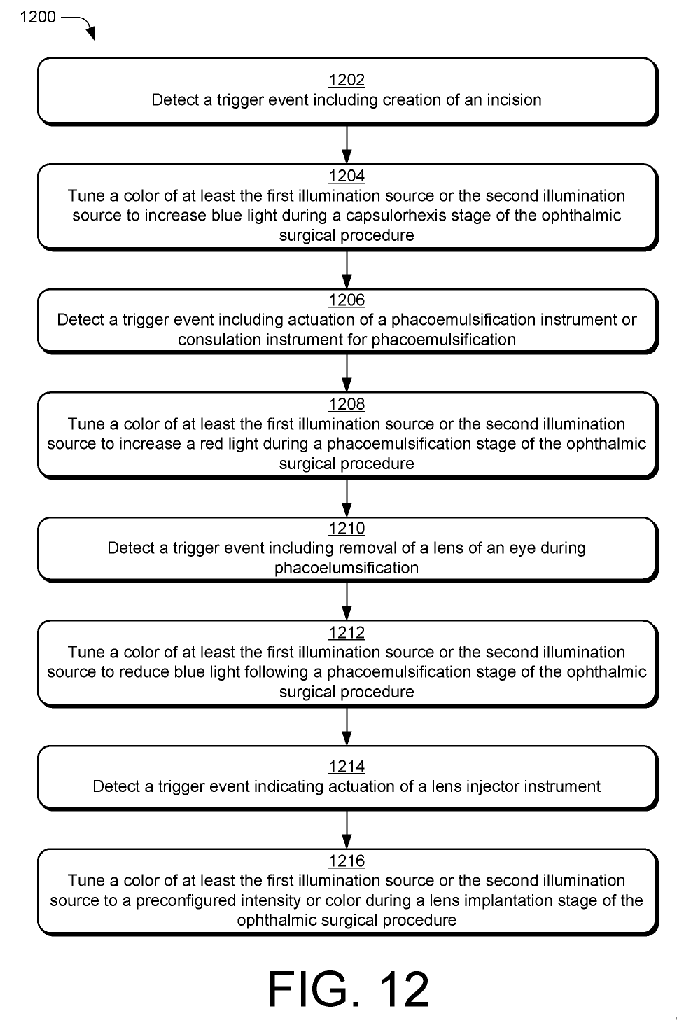

FIG. 12 depicts an example procedure 1200 in which adjusting illumination sources based on stages of ophthalmic surgery is performed during a cataract surgery.

A trigger event including creation of a corneal incision 604 is detected (block 1202). For example, the corneal incision 604 is detected based on actuation of an instrument 118 associated with performing the corneal incision 604 of the ophthalmic surgical procedure. Additionally or alternatively, some examples further comprise determining the detected trigger event based on the image feed 308 of the ophthalmic surgical procedure. For example, a machine learning model is used to identify an instrument 118 associated with performing the corneal incision 604 of the ophthalmic surgical procedure in the image feed.

A color of at least the first illumination source 122 or the second illumination source 126 is tuned to increase blue light during a capsulorhexis stage 602 of the ophthalmic surgical procedure (block 1204). In an example, the blue light is preassigned to the capsulorhexis stage 602 of the ophthalmic surgical procedure.

A trigger event including actuation of a phacoemulsification instrument 704 is detected (block 1206). For example, the phacoemulsification is detected based on actuation of an instrument associated with performing the phacoemulsification stage 702 of the ophthalmic surgical procedure. Additionally or alternatively, some examples further comprise determining the detected trigger event based on the image feed 308 of the ophthalmic surgical procedure. For example, a machine learning model is used to

identify an instrument 118 associated with performing the phacoemulsification stage 702 of the ophthalmic surgical procedure in the image feed 308.

A color of at least the first illumination source 122 or the second illumination source 126 is tuned to increase a red light during a phacoemulsification stage 702 of the ophthalmic surgical procedure (block 1208). In an example, the red light is preassigned to the phacoemulsification stage 702 of the ophthalmic surgical procedure.

A trigger event including removal of a lens of an eye during phacoemulsification is detected (block 1210). For example, the phacoemulsification stage 702 is detected based on actuation of an instrument 118 associated with performing the phacoemulsification stage 702 of the ophthalmic surgical procedure. Additionally or alternatively, some examples further comprise determining the detected trigger event based on the image feed 308 of the ophthalmic surgical procedure. For example, a machine learning model is used to identify an instrument 118 associated with performing the phacoemulsification stage 702 of the ophthalmic surgical procedure in the image feed 308.

A color of at least the first illumination source 122 or the second illumination source 126 is tuned to reduce blue light following a phacoemulsification stage 702 of the ophthalmic surgical procedure (block 1212). In an example, the reduced blue light is preassigned to the post-phacoemulsification stage of the ophthalmic surgical procedure.

A trigger event indicating actuation of a lens implantation instrument 906 is detected (block 1214). For example, the lens implantation stage 902 is detected based on actuation of an instrument 118 associated with performing the lens implantation stage 902 of the ophthalmic surgical procedure. Additionally or alternatively, some examples further comprise determining the detected trigger event based on the image feed 308 of the ophthalmic surgical procedure. For example, a machine learning model is used to identify an instrument 118 associated with performing the lens implantation stage 902 of the ophthalmic surgical procedure in the image feed 308.

A color of at least the first illumination source 122 or the second illumination source 126 is tuned to a preconfigured intensity or color during a lens implantation stage 902 of the ophthalmic surgical procedure (block 1216). In an example, the preconfigured intensity or color light is preassigned to the lens implantation stage 902 of the ophthalmic surgical procedure.

Having described example procedures in accordance with one or more implementations, consider now an example system and device that can be utilized to implement the various techniques described herein.

### Example System and Device

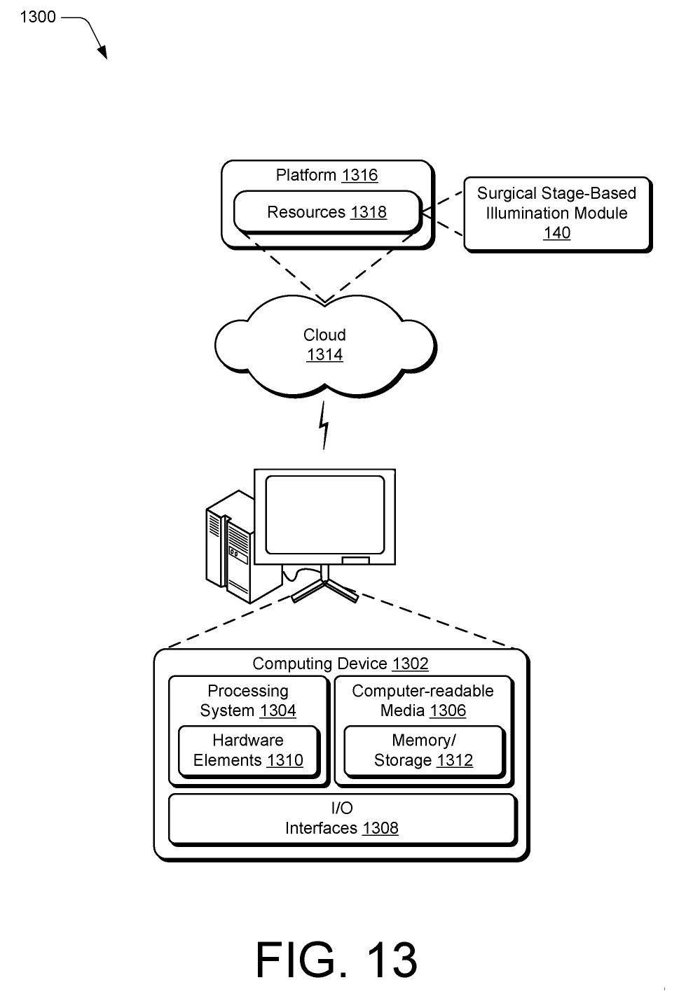

FIG. 13 illustrates an example system generally at 1300 that includes an example computing device 1302 that is representative of one or more computing systems and/or devices that may implement the various techniques described herein. The computing device 1302 may be, for example, a server of a service provider, a device associated with a client (e.g., a client device), an on-chip system, and/or any other suitable computing device or computing system. The computing device 1302 is one example of the computing system 136 of FIG. 1, for example.

The example computing device 1302 as illustrated includes a processing system 1304, one or more computer-readable media 1306, and one or more I/O interfaces 1308 that are communicatively coupled, one to another. Although not shown, the computing device 1302 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1304 is representative of functionality to perform one or more operations using hardware. Accordingly, the processing system 1304 is illustrated as including hardware elements 1310 that may be configured as processors, functional blocks, and so forth. This may include implementation in hardware as an application specific integrated circuit or other logic device formed using one or more semiconductors. The hardware elements 1310 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 1306 is illustrated as including memory/storage 1312. The memory/storage 1312 represents memory/storage capacity associated with one or more computer-readable media. The memory/storage 1312 may include volatile media (such as random-access memory (RAM)) and/or nonvolatile media (such as read only memory (ROM), Flash memory, optical disks, magnetic disks, and so forth). The memory/storage 1312 may include fixed media (e.g., RAM, ROM, a fixed hard drive, and so on) as well as removable media (e.g., flash memory, a removable hard drive, an optical disc, and so forth). The computer-readable media 1306 may be configured in a variety of other ways as further described below.

Input/output interface(s) 1308 are representative of functionality to allow a user to enter commands and information to computing device 1302, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1302 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hardwired device that performs operations based on hardwired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1302. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1302, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1310 and computer-readable media 1306 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1310. The computing device 1302 may be configured to implement particular instructions and/or functions corresponding to the software and/or hardware modules. Accordingly, implementation of a module that is executable by the computing device 1302 as software may be achieved at least partially in hardware, e.g., through use of computer-readable storage media and/or hardware elements 1310 of the processing system 1304. The instructions and/or functions may be executable/operable by one or more articles of manufacture (for example, one or more computing devices 1302 and/or processing systems 1304) to implement techniques, modules, and examples described herein.

The techniques described herein may be supported by various configurations of the computing device 1302 and are not limited to the specific examples of the techniques described herein. This functionality may also be implemented all or in part through use of a distributed system, such as over a “cloud” 1114 via a platform 1316 as described below.

The cloud 1314 includes and/or is representative of a platform 1316 for resources 1318, which are depicted including the illumination adjustment 138 in the present example. It is to be appreciated, however, that in other implementations, the illumination adjustment 138 may run locally on a client server or computer rather than on the platform 1316. The platform 1316 abstracts underlying functionality of hardware (e.g., servers) and software resources of the cloud 1314. The resources 1318 may include applications and/or data that can be utilized while computer processing is executed on servers that are remote from the computing device 1302. Resources 1318 can also include services provided over the Internet and/or through a subscriber network, such as a cellular or Wi-Fi network.

The platform 1316 may abstract resources and functions to connect the computing device 1302 with other computing devices. The platform 1316 may also serve to abstract scaling of resources to provide a corresponding level of scale to encountered demand for the resources 1318 that are implemented via the platform 1316. Accordingly, in an interconnected device example, implementation of functionality described herein may be distributed throughout the system 1300. For example, the functionality may be implemented in part on the computing device 1302 as well as via the platform 1316 that abstracts the functionality of the cloud 1314.

### Conclusion

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. An ophthalmic surgical system comprising:
a camera to obtain image feed of a surgical region of an eye, upon which an ophthalmic surgical procedure is being performed;
a first illumination source configured to produce a first light beam for illuminating the surgical region of the eye from a first direction;
a second illumination source configured to produce a second light beam for illuminating the surgical region of the eye from a second direction; and
a surgical stage-based illumination module implemented in a non-transitory computer-readable storage medium and configured to perform operations comprising:
determining, in real-time, a stage of the ophthalmic surgical procedure based on a detected trigger event; and
outputting, in real-time, instructions to adjust at least one of the first illumination source or the second illumination source based on the stage of the ophthalmic surgical procedure.

2. The ophthalmic surgical system of claim 1, wherein the first light beam is an oblique beam directed at a diagonal angle toward a cornea of the eye.

3. The ophthalmic surgical system of claim 1, wherein the second light beam includes two coaxial beams directed at a perpendicular angle toward a cornea of the eye, relative to a corneal plane of the eye.

4. The ophthalmic surgical system of claim 1, wherein the detected trigger event includes actuation of an instrument associated with performing the stage of the ophthalmic surgical procedure.

5. The ophthalmic surgical system of claim 1, wherein:
the ophthalmic surgical procedure is cataract surgery;
the detected trigger event includes creation of an incision into a cornea of the eye; and
the surgical stage-based illumination module is further configured to tune a color of at least the first illumination source or the second illumination source to increase blue light during a capsulorhexis stage of the ophthalmic surgical procedure.

6. The ophthalmic surgical system of claim 1, wherein:
the ophthalmic surgical procedure is cataract surgery;
the detected trigger event includes actuation of a phacoemulsification instrument for phacoemulsification; and
the surgical stage-based illumination module is further configured to tune a color of at least the first illumination source or the second illumination source to increase red content of light during a phacoemulsification stage of the ophthalmic surgical procedure.

7. The ophthalmic surgical system of claim 1, wherein:
the ophthalmic surgical procedure is cataract surgery;
the detected trigger event includes removal of a lens of the eye during phacoemulsification; and
the surgical stage-based illumination module is further configured to tune a color of at least the first illumination source or the second illumination source to reduce blue light following a phacoemulsification stage of the ophthalmic surgical procedure.

8. The ophthalmic surgical system of claim 1, wherein:
the ophthalmic surgical procedure is cataract surgery;
the detected trigger event includes actuation of a lens implantation instrument; and
the surgical stage-based illumination module is further configured to tune a color of at least the first illumination source or the second illumination source to a preconfigured intensity or color during a lens implantation stage of the ophthalmic surgical procedure.

9. The ophthalmic surgical system of claim 1, wherein the surgical stage-based illumination module is configured to determine the detected trigger event based on the image feed of the ophthalmic surgical procedure.

10. The ophthalmic surgical system of claim 1, wherein:
the ophthalmic surgical procedure is glaucoma surgery;
the detected trigger event includes detected use of a stent implanter instrument; and
the surgical stage-based illumination module is further configured to actuate the first illumination source, which is an oblique beam, during a stent implantation stage of the ophthalmic surgical procedure.

11. The ophthalmic surgical system of claim 1, wherein the surgical stage-based illumination module is configured to adjust a warmth of a color of the first illumination source or the second illumination source by tuning colors of the first illumination source or the second illumination source based on the stage of the ophthalmic surgical procedure.

12. The ophthalmic surgical system of claim 1, wherein the surgical stage-based illumination module is configured to tune an intensity of the first illumination source or the second illumination source based on the stage of the ophthalmic surgical procedure.

13. A method for an ophthalmic surgical procedure, comprising:
receiving, via a surgical stage-based detection system, information indicating occurrence of a trigger event during the ophthalmic surgical procedure;
determining, in real-time, a stage of the ophthalmic surgical procedure based on the trigger event; and
outputting, in real-time, instructions to adjust an oblique beam of an illumination device or coaxial beams of the illumination device based on the stage of the ophthalmic surgical procedure.

14. The method of claim 13, wherein the trigger event includes actuation of an instrument for performing the stage of the ophthalmic surgical procedure.

15. The method of claim 13, further comprising determining the trigger event based on received image feed of the ophthalmic surgical procedure.

16. The method of claim 13, further comprising adjusting a warmth of a color of the oblique beam of the illumination device or the coaxial beams of the illumination device by tuning colors of the oblique beam of the illumination device or the coaxial beams of the illumination device based on the stage of the ophthalmic surgical procedure.

17. The method of claim 13, further comprising adjusting a level of zoom of image feed of the ophthalmic surgical procedure based on the stage of the ophthalmic surgical procedure.

18. An ophthalmic surgical system comprising:
a first illumination source configured to produce an oblique beam for illuminating a surgical region of an eye, upon which an ophthalmic surgical procedure is being performed;
a second illumination source configured to produce coaxial beams for illuminating the surgical region of the eye; and
a surgical stage-based illumination module implemented in a non-transitory computer-readable storage medium and configured to perform operations comprising:
determining, in real-time, a stage of the ophthalmic surgical procedure based on a detected trigger event; and
outputting, in real-time, instructions to adjust the first illumination source or the second illumination source based on the stage of the ophthalmic surgical procedure.

19. The ophthalmic surgical system of claim 18, wherein the detected trigger event includes actuation of an instrument for performing the stage of the ophthalmic surgical procedure.

20. The ophthalmic surgical system of claim 18, wherein the surgical stage-based illumination module is configured to determine the detected trigger event based on image feed of the ophthalmic surgical procedure.
